(19) United States
(12) Patent Application Publication
(10) Pub. No.: US 2026/0158511 A1
(43) Pub. Date: Jun. 11, 2026

(54) FLUID DISPENSING DEVICE AND ASSEMBLY METHOD THEREFOR

(71) Applicant: Aero Pump GmbH, Hochheim/Main (DE)

(72) Inventors: Sebastian ROTHER, Mainz (DE); Milena MARSZALEK, Mainz (DE); Elena DE TOMASO, Ginsheim (DE)

(73) Assignee: Aero Pump GmbH, Hochheim/Main (DE)

(21) Appl. No.: 19/368,671

(22) Filed: Oct. 24, 2025

(30) Foreign Application Priority Data
Dec. 6, 2024 (DE) 20 2024 107 072

(51) Int. Cl. B05B 11/10; A61M 15/00; A61M 15/08

(52) U.S. Cl. CPC B05B 11/1004; A61M 15/009; A61M 15/08; B05B 11/1016

(57) ABSTRACT

A fluid dispensing device and method of assembling a fluid dispensing device. The device includes a reservoir and a pump system, in which the pump system has at least one outlet opening and a sleeve that is movable relative to the reservoir in order to dispense fluid from the reservoir through the outlet opening. The reservoir has an outer wall, in which the sleeve is guided in a sealing manner, and a mandrel extending in the direction of the sleeve. The sleeve has a first section with a channel and a second section with a receptacle adapted to the outer contour of the mandrel, such that at least one fluid channel is formed between the mandrel and the receptacle.

### CROSS-REFERENCE TO RELATED APPLICATION

This application claims priority under 35 U.S.C. § 119(a) to German Utility Model No. 20 2024 107 072 filed Dec. 6, 2024, the disclosure of which is expressly incorporated by reference herein in its entirety.

### BACKGROUND

### 1. Field of the Invention

Embodiments are directed to a fluid dispensing device with a pump system and a reservoir and to a method for assembling a fluid dispensing device. The pump system may have at least one outlet opening and a sleeve that is movable relative to the reservoir in order to dispense fluid from the reservoir through the outlet opening.

### 2. Discussion of Background Information

Such fluid dispensing devices are often hand-operated pumps, in particular spray or drip pumps, for example, for nasal, inhalative, buccal, otological, topical, or ophthalmological administration of pharmaceutical or cosmetic liquids. While such fluid dispensing devices are usually intended for dispensing multiple doses of the fluid, there are also single-use dispensers for medical or cosmetic applications in which only a single dose of the fluid is to be dispensed. Examples of this are described in DE 10 2014 003 622 B4 or EP 3 727 534 B1.

It has been found that known systems sometimes require complex assembly procedures in order to fill the fluid in a microbiologically sealed manner. In addition, the residual volume of fluid that remains unused after use in known disposable dispensers is sometimes relatively high due to their design, which is unsatisfactory, especially in the case of expensive medications. In some cases, protection against unintentional use is also considered to be in need of improvement.

### SUMMARY

Embodiments of the present invention provide a fluid dispensing device and an assembly method therefor which avoid the above-mentioned disadvantages of known systems.

According to embodiments, a fluid dispensing device and a method for assembling a fluid dispensing device include the features recited in the claims.

In embodiments, a fluid dispensing device includes a pump system and a reservoir, in which the pump system may have at least one outlet opening and a sleeve that is movable relative to the reservoir in order to dispense fluid from the reservoir through the outlet opening. The sleeve can plunge into the reservoir. To minimize the amount of fluid remaining in the fluid dispensing device after a single use, the sleeve and the reservoir are preferably adapted to each other. If the reservoir has, for example, a roughly cylindrical outer wall in which the sleeve is guided in a sealing manner, and a mandrel extending from the bottom of the reservoir towards the sleeve, then the sleeve may, for example, have a first section with a channel and a second section with a receptacle adapted, e.g., partially cylindrical, to the outer contour of the mandrel with at least one fluid channel formed between the mandrel and the receptacle. In other words, the contour of the receptacle can be adapted to the mandrel of the reservoir in such a way that they fit together and the dead volume is thus limited to the volume of the fluid channel. This minimizes the dead volume remaining in the reservoir due to its design. The fluid channel also serves to prevent any blockages in the pump due to the tight fits. Such a fluid dispensing device according to the invention also has the advantage that it can be used in any orientation in space because the sleeve dispenses fluid from the reservoir like a syringe plunger.

In order to minimize the dead volume remaining in the reservoir due to the design, the sleeve and the mandrel can be adapted to each other in such a way that there is essentially no gap between the sleeve and the mandrel and that they fit together snugly apart from the fluid channel. However, a small gap between the mandrel and the sleeve may also be provided, which may be advantageous due to manufacturing tolerances. Embodiments therefore also included in which there is a small gap between the sleeve and the mandrel. However, this gap should preferably be as small as possible.

According to a preferred embodiment, the mandrel and the receptacle may each have a conical section. In particular, the tip of the mandrel facing the pump system may be tapered, for example, as a cone tip or a ball tip, whereby the receptacle has a congruent inner contour as a transition to the channel, the inner diameter of which is preferably smaller than that of the cylindrical part of the receptacle.

The fluid channel can be formed either by at least one groove in the mandrel and/or by at least one groove in the receptacle. The groove preferably runs in the axial direction, i.e., parallel to the central axis of the fluid dispensing device. This minimizes the volume of the fluid channel and thus the dead volume of the fluid dispensing device.

Irrespective of this, the dead volume can also be further reduced by the channel of the sleeve having a smaller inner diameter than the receptacle and/or being shorter than the receptacle.

The pump system of the fluid dispensing device may further include a valve arrangement and/or a cylinder which is in fluid communication with the channel of the sleeve. The valve arrangement may be formed by a closure element which is guided in a displacement manner in a cavity of the cylinder against the force of a spring between a position closing a fluid opening and a position releasing the fluid opening. This includes designs in which the spring is formed integrally with the closure element and/or the closure element itself is at least partially elastically deformable. In addition, the fluid dispensing device, in particular the pump system, may further comprise a head part for receiving the cylinder, wherein the at least one outlet opening is provided in the head part. A fluid channel connecting the fluid opening to the outlet opening may be formed in the cylinder and/or the head part. In particular, this fluid channel may be formed by a groove in the outer surface of the cylinder and/or a groove in the inner surface of the head part.

The reservoir can be guided in a displaceable manner in the head part, in particular for a defined distance corresponding to an application stroke. The maximum stroke can, for example, be limited by a stop on the sleeve at the bottom of the reservoir. In this case, the reservoir can be held securely in the head part by means of a locking mechanism. In particular, the reservoir and the head part are connected to each other by means of undercut areas in such a way that non-destructive disassembly of the fluid dispensing device can be avoided as far as possible.

In order to prevent unintentional actuation of the fluid dispensing device, the reservoir may, for example, have a removable actuation protection element. The actuation protection element may be designed as a cap covering part of the reservoir, which has a stop designed in such a way that it prevents or at least limits movement of the reservoir towards the pump system.

The fluid dispensing device may additionally have an optional protective cap to prevent contamination of the head section. Furthermore, the head section may optionally have a radially protruding flange and/or a radially protruding flange-like finger wing may be provided on the head section to facilitate operation of the fluid dispensing device.

According to other embodiments, a method for assembling a fluid dispensing device, in particular a fluid dispensing device of the type described above, includes: providing a reservoir, which is partially accommodated in a removable actuation protection element, and a pump system; filling the reservoir with a fluid, in particular with a liquid active ingredient; placing and locking/engaging the pump system onto the reservoir, whereby the actuation protection element and the pump system are positioned against each other in such a way that movement of the reservoir toward the pump system is prevented. It is particularly preferred if the pump system is manufactured according to a modular system in which individual parts are inserted into each other and connected to each other with form-fit or press-fit connections. The pump system can thus be assembled by inserting a spring and a closure element into a cylinder and by inserting the cylinder and a sleeve into a head part. This allows all components to be assembled solely by axial movement parallel to the central axis of the fluid dispensing device.

Embodiments are directed to a fluid dispensing device that includes a reservoir; and a pump system. The pump system includes at least one outlet opening and a sleeve that is movable relative to the reservoir in order to dispense fluid from the reservoir through the outlet opening, and the reservoir includes an outer wall, in which the sleeve is guided in a sealing manner, and a mandrel extending in the direction of the sleeve. The sleeve includes a first section with a channel and a second section with a receptacle adapted to the outer contour of the mandrel, such that at least one fluid channel is formed between the mandrel and the receptacle.

According to embodiments, the mandrel and the receptacle may each have a conical section. Alternatively, or additionally, the mandrel and the receptacle can each have a cylindrical section.

In embodiments, the fluid channel can be formed by at least one groove in the mandrel. Alternatively, the fluid channel may be formed by at least one groove in the receptacle of the sleeve.

In accordance with embodiments, the channel of the sleeve can have a smaller inner diameter than the receptacle and/or may be shorter than the receptacle.

According to other embodiments, the pump system may include a cylinder in which a closure element is guided to be movable against a force of a spring between a position closing an opening and a position opening the opening, the cylinder being connected to the channel of the sleeve for fluid flow. The pump system may further include a head part for receiving the cylinder, in which the outlet opening is provided, such that a fluid channel can be formed in the cylinder and/or the head part may have a fluid channel connecting the opening to the outlet opening. Further, the reservoir may be guided in a displaceable manner in the head part. Still further, the reservoir can be snapped in the head part.

In other embodiments, the reservoir can have a removable actuation protection element. The actuation protection element may be designed as a cap that partially covers the reservoir and can have a stop that is designed to prevent or limit movement of the reservoir toward the pump system.

In still other embodiments, the head part may have a radially protruding flange, and/or a radially protruding flange-like finger wing may be provided on the head part.

Embodiments are directed to a method for assembling the fluid dispensing device discussed above. The method includes providing the reservoir partially accommodated in a removable actuation cover protection element and the pump system; filling the reservoir with a fluid; and placing and engaging the pump system onto the reservoir. The removable actuation protection element and the pump system are positioned against each other in such a way that movement of the reservoir in the direction of the pump system is prevented.

In further embodiments, the method can include assembling the pump system by inserting a spring and a closure element into a cylinder and by inserting the cylinder and a sleeve into a head part.

According to other embodiments, the fluid may include a liquid active ingredient.

Embodiments are directed to a method for assembling a fluid dispensing device. The method includes providing a reservoir that is partially accommodated in a removable actuation cover protection element and a pump system; filling the reservoir with a fluid; placing and engaging the pump system onto the reservoir. The removable actuation protection element and the pump system are positioned against each other in such a way that movement of the reservoir in the direction of the pump system is prevented.

In accordance with still other embodiments, the method can include assembling the pump system by inserting a spring and a closure element into a cylinder and by inserting the cylinder and a sleeve into a head part. Moreover, the fluid may include a liquid active ingredient.

Other exemplary embodiments and advantages of the present invention may be ascertained by reviewing the present disclosure and the accompanying drawing.

### BRIEF DESCRIPTION OF THE DRAWINGS

The invention is described below with reference to preferred embodiments in conjunction with the drawings. These show schematically:

FIG. 1 is a sectional view of a fluid dispensing device according to a first embodiment of the invention before use;

FIG. 2 is a sectional view of the fluid dispensing device according to FIG. 1 after use;

FIG. 3 is an enlarged sectional view showing a detail of the fluid dispensing device according to FIG. 1 after use;

FIG. 4 is an enlarged section along line IV-IV in FIG. 3;

FIG. 5 is an enlarged sectional view showing a detail of the fluid dispensing device according to a second embodiment of the invention after use;

FIG. 6 is an enlarged view of a section along line VI-VI in FIG. 5; and

FIG. 7 shows a sectional view of a fluid dispensing device according to a third embodiment of the invention before use.

### DETAILED DESCRIPTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

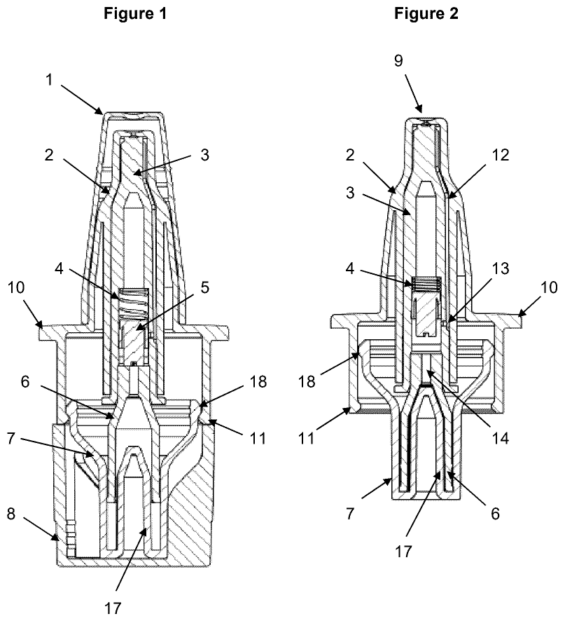

The fluid dispensing device shown in FIG. 1 in a state prior to initial use essentially comprises a protective cap 1, a head part 2 (base part), a cylinder 3 (liner), a spring 4, a closure element 5 designed as a piston, a sleeve 6, a reservoir 7, and an actuation protection element 8. In addition, a finger wing (not shown) may be provided.

The removable protective cap 1 encloses the head part 2 and is removed before use of the fluid dispensing device. In the shown state, it prevents contamination of the head part 2.

The head part 2 is essentially designed as a sleeve that encloses a cavity for receiving the cylinder 3. At the upper end shown in the figure, the head part 2 has an outlet opening 9. In the embodiment shown, the head part 2 has a contour for inserting the head into the nose. Together with the liner or cylinder 3, the head part 2 forms, in the exemplary illustrated embodiment, a turbulence chamber in order to atomize the fluid. If the fluid dispensing device is not designed as a nasal pump or spray pump, the contour may be designed differently. A radially protruding flange 10 is formed on the head part 2 as a finger rest. In addition, a finger wing (not shown) that protrudes even further radially can rest on this flange 10. The finger wing is intended to widen the support surface for the fingers and can prevent slipping with grip grooves. This is intended to facilitate operation. In the figure, a cylindrical section with an inwardly directed locking bead 11 extends below the flange 10. This lower edge of the head part 2 also forms a stop surface for the actuation protection element 8.

The cylinder 3 is inserted into the cavity of the head part 2 and, in the example shown, also has a taper in the upper area of the figure. The cylinder 3 directs the fluid via an external fluid channel 12, which is closed with the head part 2, into the turbulence chamber, which is finally discharged via the outlet opening 9. The fluid channel 12 can be formed, for example, by a groove approximately 0.2 mm to 1.0 mm deep, in particular approximately 0.4 mm deep, in the outer surface of the cylinder. Alternatively, or additionally, such a fluid channel can also be formed by a corresponding recess in the inner surface of the head part 2. The fluid channel 12 is connected to a cavity inside the cylinder 3 via a lateral opening 13. This cavity has a shoulder on which the spring 4 is supported.

Spring 4 is inserted into the cavity of the cylinder together with the piston forming a closure element 5, so that the piston can be moved upward against the force of spring 4 in the figure. The spring 4 pushes the piston into the position shown in FIG. 1, in which the piston can seal the opening 13. This allows pressure to build up and the pump chamber to be closed after use. The piston (sealing/closure element 5) is used, among other things, to seal the pump. In particular, it prevents the fluid from escaping and contaminating the environment without being activated. In the example shown, the piston has a circumferential sealing lip with which it seals against the inner wall of the cylinder 3.

The sleeve 6 has a first, cylindrically shaped section (upper end in the figures) in which a channel 14 is formed. This first section is pressed into the cavity of the cylinder 3 in a sealing manner. A second section of sleeve 6, shown at the bottom of the figures, has a larger outer diameter than the first section and is also cylindrical in some areas. The second section is conical in shape at the transition to the first section. The sleeve 6 is guided in a sealed manner in the reservoir 7. For this purpose, a sealing lip 15 and a guide ring 16 are provided on the outer surface of the sleeve 6. The inner contour of the second section of the sleeve 6 is precisely adapted to the outer contour of a mandrel 17 of the reservoir 7.

The reservoir 7 serves as a container for the fluid. It has a region with a cylindrical outer wall (lower region in the figures) and a region with a widening outer diameter with a locking bead 18. The mandrel 17 protrudes from the bottom of the reservoir 7 towards the sleeve 6. This creates an annular space between the outer wall and the mandrel 17, which is filled with fluid in FIG. 1 and in which the second section of the sleeve 6 is guided. In the example shown, the tip of the mandrel 17 is conical in shape to match the inner contour of the sleeve 6.

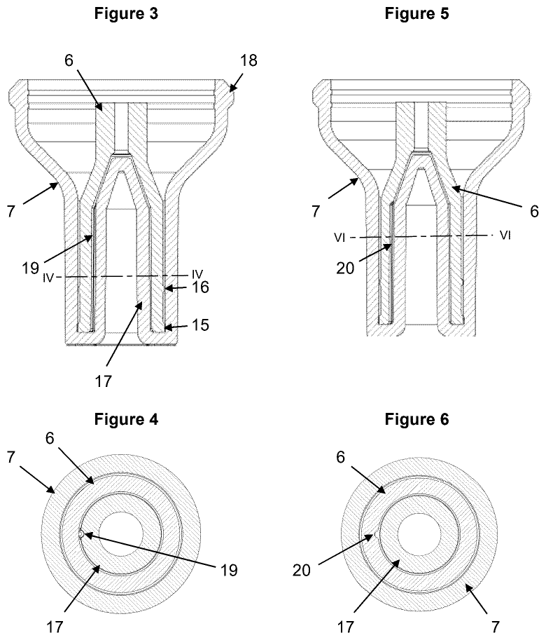

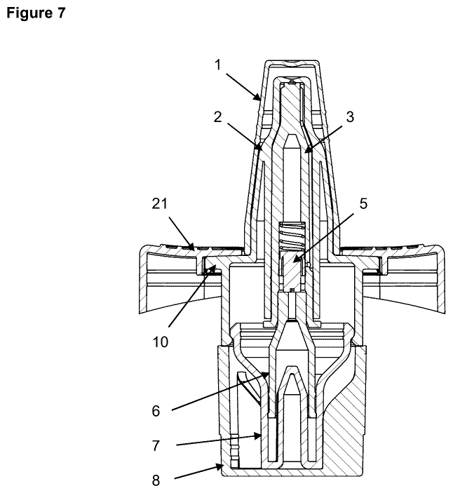

As can be seen from FIGS. 3 and 4, in the first embodiment, a fluid channel 19 is formed as a groove in the outer surface of the mandrel 17. In contrast, in the second embodiment shown in FIGS. 5 and 6, a fluid channel 20 is formed as a groove in the inner surface of the second section of the sleeve 6. In both cases, this groove is only very small, for example only 0.2 mm to 1.0 mm, in particular less than 0.3 mm, deep, in order to minimize the dead volume.

The removable actuation protection element 8 is intended to prevent unintentional actuation of the pump. This rests with an upper edge in the figures on the lower edge of the head part 2. If the actuation protection element 8 is present as shown in FIG. 1, it surrounds the reservoir 7 so that it cannot move relative to the sleeve 6. The pump can only be activated after the actuation protection element 8 has been removed.

The fluid dispensing device is filled by first inserting the reservoir 7 into the actuation protection element 8 and filling the annular space between the outer wall and the mandrel 17 with a certain amount of the product. The remaining components, namely the head part 2, the cylinder 3, the spring 4, the closure element (piston) 5, and the sleeve 6, are preassembled into a pump assembly, in particular by inserting them into one another and, insofar as they are not to be movable relative to one another, connecting them to one another with form fits or press fits.

This pump assembly is then snapped onto the assembly including reservoir 7 and actuation protection element 8. The stop of the actuation protection element 8 with the head part 2 ensures the correct position of the reservoir 7. The locking bead 11 and the locking bead 18 interlock and prevent the reservoir 7 from being pulled out of the head part 2 when the actuation protection element 8 is removed. The function of the actuation protection element 8 is to hold the reservoir 7 securely in place during the filling phase by means of webs and to guide the reservoir 7 correctly into the head part 2. The stop of the actuation protection element 8 to the head part 2 is designed so that the reservoir 7 is pushed into the head part 2 without the risk of the product flowing out prematurely. To operate the pump, the actuation protection element 8 must then be pulled off, but the reservoir 7, which is held in the head part 2 by the undercut of the locking beads 11 and 18, must remain in its position. This means that the removal force of the actuation protection element 8 should preferably be lower than the removal force of the reservoir 7 from the head part 2.

The following section explains how the pump works, which is identical for both embodiments. To operate the pump, the user must first remove the protective cap 1 and the actuation protection element 8.

The pump is then inserted into the nose, for example, and activated by pressing the reservoir 7 against the sleeve 6 in the direction of the head part 2. The sleeve 6 is sealed in the reservoir 7 and the volume in the reservoir 7 is reduced. This forces the liquid inside to move through fluid channels 19 and 20, respectively, and channel 14 towards piston 5. When a defined pressure is reached, the piston moves against the force of spring 4, thereby opening the opening 13 so that the liquid is introduced into the turbulence chamber through fluid channel 12. A comparison of the position of the piston of the closure element 5 in FIGS. 1 and 2 shows the displacement of the piston and the compression of the spring 4, so that the opening 13 in FIG. 1 is closed by the piston and is released in FIG. 2. After turbulation, the fluid leaves the pump in atomized form through the outlet opening 9.

After a complete stroke of the reservoir 7 relative to the sleeve 6, these collide with each other in the area of the bottom of the reservoir 7, as shown in FIG. 2, whereby the reservoir 7 is emptied as far as possible, i.e., up to the dead volume remaining in the fluid channel 19 or 20 due to the design. A small amount of fluid that cannot be discharged also remains in the channel 14, the opening 13, and the fluid channel 12.

The design shown of the reservoir 7 and the sleeve 6 with only one fluid channel 19 or 20 and the conical contours on the mandrel 17 and the sleeve 6 means that, for example, the dead volume that remains after a stroke of the reservoir 7 as described above in the space between the reservoir 7 and the sleeve 6, in particular, in the fluid channel 19 or 20, is less than 10%, in particular less than 5%, for example approximately 4%, of the volume to be dispensed.

In addition, the design of reservoir 7 shown in the figures, with a mandrel 17 formed in the base and a widening area with the locking bead 18, allows the reservoir to be manufactured as a plastic injection-molded part. Preferably, the other components of the fluid dispensing device according to the invention, with the possible exception of spring 4, are also plastic injection molded parts.

The invention is shown in the figures as an example of a nasal spray as a disposable dispenser, i.e., with a single-dose pump, with which the contents of the reservoir 7 are dispensed in a single stroke. However, the advantages of the low dead volume can also be exploited in the same way for other types of pumps, for example for dropper pumps in which the fluid is not atomized, or for inhalative, buccal, otological, topical, or ophthalmological administrations. In these cases, the head part 2 in particular may have a shape that differs from that shown in the figures.

FIG. 7 shows a fluid dispensing device similar to the view in FIG. 1, but with an additional finger wing 21 adjacent to the flange 10 to facilitate actuation.

According to another embodiment, a method for assembling a fluid dispensing device, in particular, a fluid dispensing device of the type described above, comprises:
providing a reservoir 7, which is partially accommodated in a removable actuation protection element 8, and a pump system,
filling the reservoir 7 with a fluid, in particular, with a liquid active ingredient,
placing and locking the pump system onto the reservoir 7, whereby the actuation protection element 8 and the pump system are positioned against each other in such a way that movement of the reservoir 7 in the direction of the pump system is prevented.

This method is optionally characterized in that the pump system is assembled by inserting a spring 4 and a closure element 5 into a cylinder 3 and by inserting the cylinder 3 and a sleeve 6 into a head part 2.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

### REFERENCE NUMBERS

1 protective cap
2 head part
3 cylinder
4 spring
5 closure element (piston)
6 sleeve
7 reservoir
8 actuation protection element
9 outlet opening
10 flange
11 locking bead
12 fluid channel
13 opening
14 channel
15 sealing lip
16 guide ring
17 mandrel
18 locking bead
19 fluid channel
20 fluid channel
21 finger wing

What is claimed:

1. A fluid dispensing device comprising:
a reservoir; and
a pump system, the pump system having at least one outlet opening and a sleeve that is movable relative to the reservoir in order to dispense fluid from the reservoir through the outlet opening,
wherein the reservoir has an outer wall, in which the sleeve is guided in a sealing manner, and a mandrel extending in the direction of the sleeve, and
wherein the sleeve has a first section with a channel and a second section with a receptacle adapted to the outer contour of the mandrel, such that at least one fluid channel is formed between the mandrel and the receptacle.

2. The fluid dispensing device according to claim 1, wherein the mandrel and the receptacle each have a conical section.

3. The fluid dispensing device according to claim 1, wherein the mandrel and the receptacle each have a cylindrical section.

4. The fluid dispensing device according to claim 1, wherein the fluid channel is formed by at least one groove in the mandrel.

5. The fluid dispensing device according to claim 1, wherein the fluid channel is formed by at least one groove in the receptacle of the sleeve.

6. The fluid dispensing device according to claim 1, wherein the channel of the sleeve has a smaller inner diameter than the receptacle and/or is shorter than the receptacle.

7. The fluid dispensing device according to claim 1, wherein the pump system comprises a cylinder in which a closure element is guided to be movable against a force of a spring between a position closing an opening and a position opening the opening, the cylinder being connected to the channel of the sleeve for fluid flow.

8. The fluid dispensing device according to claim 7, wherein the pump system further comprises a head part for receiving the cylinder, in which the outlet opening is provided, wherein a fluid channel is formed in the cylinder and/or the head part has a fluid channel connecting the opening to the outlet opening.

9. The fluid dispensing device according to claim 8, wherein the reservoir is guided in a displaceable manner in the head part.

10. The fluid dispensing device according to claim 8, wherein the reservoir is snapped in the head part.

11. The fluid dispensing device according to claim 1, wherein the reservoir has a removable actuation protection element.

12. The fluid dispensing device according to claim 11, wherein the actuation protection element is designed as a cap that partially covers the reservoir and has a stop that is designed to prevent or limit movement of the reservoir toward the pump system.

13. The fluid dispensing device according to claim 8, wherein the head part has a radially protruding flange, and/or in that a radially protruding flange-like finger wing is provided on the head part.

14. A method for assembling the fluid dispensing device according to claim 1, the method comprising:
providing the reservoir partially accommodated in a removable actuation cover protection element and the pump system;
filling the reservoir with a fluid; and
placing and engaging the pump system onto the reservoir, whereby the removable actuation protection element and the pump system are positioned against each other in such a way that movement of the reservoir in the direction of the pump system is prevented.

15. The method according to claim 14, further comprising assembling the pump system by inserting a spring and a closure element into a cylinder and by inserting the cylinder and a sleeve into a head part.

16. The method according to claim 14, wherein the fluid comprises a liquid active ingredient.

17. A method for assembling a fluid dispensing device, the method comprising:
providing a reservoir that is partially accommodated in a removable actuation cover protection element and a pump system;
filling the reservoir with a fluid;
placing and engaging the pump system onto the reservoir, whereby the removable actuation protection element and the pump system are positioned against each other in such a way that movement of the reservoir in the direction of the pump system is prevented.

18. The method according to claim 17, further comprising assembling the pump system by inserting a spring and a closure element into a cylinder and by inserting the cylinder and a sleeve into a head part.

19. The method according to claim 17, wherein the fluid comprises a liquid active ingredient.
